(19) United States
(12) Patent Application Publication
(10) Pub. No.: US 2026/0341420 A1
(43) Pub. Date: Oct. 29, 2026

(54) GENERATING BREAKDOWN PRODUCTS IN BIODEGRADATION

(71) Applicant: BASF SE, Ludwigshafen am Rhein (DE)

(72) Inventors: Jessica Eleanor Mugleston, Stockport Greater Manchester (GB); Volker Settels, Ludwigshafen am Rhein (DE); Soumya Daturpalli, Ludwigshafen am Rhein (DE); Glauco Battagliarin, Ludwigshafen am Rhein (DE); Frederik Sandfort, Ludwigshafen am Rhein (DE); Averina Nicolae, Ludwigshafen am Rhein (DE)

(21) Appl. No.: 19/472,849

(22) PCT Filed: Apr. 5, 2024
(86) PCT No.: PCT/EP2024/059392

§ 371 Date: Oct. 6, 2025

(30) Foreign Application Priority Data
Apr. 6, 2023 (EP) 23167119.9

(51) Int. Cl. G16C 20/20; G16C 20/10; G16C 20/70

(52) U.S. Cl. CPC G16C 20/20; G16C 20/10; G16C 20/70

(57) ABSTRACT

A computer implemented method for generating breakdown products of a chemical material comprising the steps of providing a target chemical material; providing a biodegradation habitat, indicative of an enzyme environment in the biodegradation habitat providing a breakdown model, relating a chemical material and the biodegradation habitat to breakdown products generating breakdown products based on the provided target chemical material and the breakdown model providing the breakdown products.

### TECHNICAL FIELD

The disclosure relates to a method, an apparatus and a computer program product for generating a measure or for measuring biodegradability of a chemical material. Further, the disclosure refers to a training method, a training apparatus and a training computer program for training a data driven biodegradation or break down model suitable for the method, apparatus and computer program product for generating a measure or for measuring biodegradability of a chemical material. Moreover, the disclosure refers to a method and apparatus for generating a target synthesis specification for a chemical material. Moreover, the disclosure relates to a method, apparatus and computer program product for generating breakdown products or data associated with breakdown products of a chemical material or molecule.

### TECHNICAL BACKGROUND

Development of new chemical materials that are tailored to application requirements is a predominant problem in modern chemical industries. Recently, a further requirement is also raised, related to the environmental impact of the chemical product along the life cycle of the chemical product. One important aspect of the environmental impact is prevention of accumulation of waste, waste can be avoided if the chemical material is biodegradable. Chemical materials are therefore not only developed in view of their technical application property but also in view of the property biodegradability. Biodegradability is a physico chemical property of the chemical product. Biodegradability of a chemical material may vary dependent on the habitat in which the chemical material is disposed.

### SUMMARY

In an aspect the disclosure relates to a computer implemented method for generating breakdown products of a chemical material comprising the steps of
providing a target chemical material;
providing a biodegradation habitat, indicative of an enzyme environment in the biodegradation habitat;
providing a breakdown model, relating a chemical material and the biodegradation habitat to breakdown products;
generating depending on the biodegradation habitat breakdown products based on the provided target chemical material provided to the breakdown model;
providing the breakdown products.

In an aspect the disclosure relates to a computer implemented method for generating biodegradation data associated with breakdown products of a chemical material, wherein the break down products relate to fragments of the chemical material on at least partial decomposition of the chemical material, the method comprising the steps of:
providing a digital representation associated with the chemical material;
providing a digital representation associated with the biodegradation habitat, wherein the digital representation of the biodegradation habitat may relate to e.g. a microbial composition of the biodegradation habitat, e.g. including at least one microbial community, including an enzyme environment or including one or more enzyme(s), one or more bacteria, one or more algae and/or one or more fungi,
providing at least one breakdown model configured to map the digital representation associated with the chemical material depending on the digital representation associated with the biodegradation habitat to biodegradation data associated with breakdown products,
generating biodegradation data associated with breakdown products by providing the digital representation associated with the chemical material to the breakdown model, in particular depending on the digital representation associated with the biodegradation habitat,
providing biodegradation data associated with breakdown products.

In another aspect, the disclosure relates to a computer implemented method for generating a measure of biodegradability or measuring biodegradation of a chemical material comprising the steps of:
providing a chemical material,
providing a biodegradation habitat indicative of an enzyme environment in the biodegradation habitat,
providing a breakdown model, relating a chemical material and the biodegradation habitat to breakdown products,
generating breakdown products based on the provided target chemical material and the breakdown model,
providing the breakdown products,
providing a biodegradation model, relating biodegradability of the breakdown products to a measure for biodegradability,
determining a measure of biodegradability of the chemical material, based on the biodegradation model and the breakdown products,
providing the measure for biodegradability.

In another aspect, the disclosure relates to an apparatus for generating a measure of biodegradability or measuring biodegradation of a chemical material comprising:
a material providing interface configured to provide a chemical material,
a habitat providing configured to provide a biodegradation habitat indicative of an enzyme environment in the biodegradation habitat,
a model providing interface configured to provide a breakdown model, relating a chemical material and the biodegradation habitat to breakdown products,
a break down product generator configured to generate breakdown products based on the provided target chemical material and the habitat biodegradation provided to the breakdown model,
an output interface configured to provide the breakdown products,
a biodegradation model providing interface configured to provide a biodegradation model, relating biodegradability of the breakdown products to a measure for biodegradability,
a determinator configured to determine a measure of biodegradability of the chemical material, based on the biodegradation model and the breakdown products,
a biodegradability output interface configured to provide the measure for biodegradability.

In another aspect, the disclosure relates to an apparatus for generating a measure of biodegradability or measuring biodegradation of a chemical material comprising the steps of:
a material providing interface configured to provide a chemical material,
a habitat providing interface configured to provide a biodegradation habitat indicative of an enzyme environment in the biodegradation habitat,
a model providing interface configured to provide a breakdown model, relating a chemical material and the biodegradation habitat to breakdown products,
a generator configured to generate breakdown products based on the provided target chemical material and the breakdown model,
a breakdown products output interface configured to provide the breakdown products,
a biodegradability model providing interface configured to provide a biodegradation model, relating biodegradability of the breakdown products to a measure for biodegradability,
a determinator configured to determine a measure of biodegradability of the chemical material, based on the biodegradation model and the breakdown products,
a biodegradability output interface configured to provide the measure for biodegradability.

In another aspect, the disclosure relates to an apparatus for generating breakdown products, wherein the apparatus comprises:
a digital representation providing unit for providing the chemical material,
a habitat providing unit for providing a biodegradation habitat, wherein the bio-degradation habitat is indicative of an enzyme environment in the biodegradation habitat
a model providing unit for providing a breakdown model habitat, wherein the biodegradation model is adapted to generate breakdown products of a chemical material in the respective biodegradation habitat, wherein the biodegradation model is a data driven model or an enzymatic pathway model, and
a generating unit for generating the breakdown products of the chemical material based on the breakdown model, the provided biodegradation habitat and the chemical material.

In an aspect the disclosure relates to an apparatus for generating biodegradation data associated with breakdown products of a chemical material, wherein the break down products relate to fragments of the chemical material on at least partial decomposition of the chemical material, the method comprising the steps of:
a material providing interface configured to providing a digital representation associated with the chemical material,
a habitat providing interface configured to providing a digital representation associated with the biodegradation habitat, wherein the digital representation of the biodegradation habitat may relate to e.g. a microbial composition of the biodegradation habitat, e.g. including at least one microbial community, including an enzyme environment or including one or more enzyme(s), one or more bacteria, one or more algae and/or one or more fungi,
a model providing interface providing at least one breakdown model configured to map the digital representation associated with the chemical material depending on the digital representation associated with the biodegradation habitat to biodegradation data associated with breakdown products,
a generation interface configured to generate biodegradation data associated with breakdown products by providing the digital representation associated with the chemical material to the breakdown model, in particular depending on the digital representation associated with the biodegradation habitat,
an output interface configured to provide biodegradation data associated with breakdown products.

In another aspect disclosed is a computer implemented method for generating a target synthesis specification, particularly for production of the chemical material and/or monitoring and/or controlling production of the chemical material, by providing biodegradation data, such as the break down products, at least one target biodegradability measure and/or at least one ecotoxicological measure and selecting one or more chemical material(s) based on the break down product generation as disclosed herein.

In another aspect, the disclosure relates to an interface apparatus for providing an interface, wherein the interface apparatus comprises:
an input interface unit for receiving as input chemical material and a habitat via a user interface and for providing the received chemical material and the biodegradation habitat indicative of an enzyme environment in the biodegradation habitat,
a result interface for providing the generated breakdown products of the chemical material to a user via a user interface as result, wherein the result is received from an apparatus for generating breakdown products.

In an aspect the disclosure relates to interface method for providing an interface, wherein the interface method comprises:
receiving as input a chemical material and a biodegradation habitat via a user interface and providing the received digital representation and the habitat to a processor performing the method disclosed herein, and
providing the generated breakdown products of the chemical material to a user via a user interface as result, wherein the result is received from the processor performing the method disclosed herein.

In yet another aspect the present disclosure relates to a computer program product with instructions, which when executed on one or more computing node(s) and/or processors is configured to carry out the steps of the method(s) of the present disclosure or configured to be carried out by the apparatus(es) of the present disclosure.

### EMBODIMENTS

Any disclosure, embodiments and examples described herein relate to the methods, the systems, apparatuses, chemical products and computer elements lined out above and below. Advantageously, the benefits provided by any of the embodiments and examples equally apply to all other embodiments and examples.

By generating the break down products the biodegradation process the chemical material will undergo in a certain habitat can be monitored more reliably on design of the polymer. In particular by generating the break down products ecotox properties during the biodegradation process can be determined even for intermediate stage of fragmentation and the biodegradability of the chemical material may be determined based on the break down products.

The chemical material may include or be any chemical molecule suitable for biodegradation in the biodegradation habitat or to be tested for biodegradation in the biodegradation habitat. The chemical material may include or be a macro molecule or a small molecule. The chemical material may include or be a polymer or a functional chemical compound(s).

In an embodiment, chemical material may refer to a product obtained by means of a chemical production process. Chemical production process may refer to a process including one or more chemical reaction(s). The chemical material may include a raw material. The chemical material may include a chemical material produced by reacting at least two raw materials. The chemical material may include a component. The chemical material may include a component assembly. The chemical material may include an end product. The chemical material may include natural chemical materials. Natural chemical materials may include any chemical material that is produced by nature without human interaction or intervention, i.e. any unprocessed chemical substance that is found in nature, such as chemicals from plants, micro-organisms, animals, the earth and the sea or any chemical substance that is found in nature and extracted using a process that does not change its chemical composition. Natural chemical materials may include biologicals like enzymes as well naturally occurring inorganic or organic chemical materials. Natural chemical materials may be isolated and purified prior to their use or they can be used in unisolated and/or unpurified form. Chemical materials may be synthetic chemical materials. Synthetic chemical materials may include chemical materials produced with human interaction or intervention. Synthetic chemical materials may be produced with the same chemical reactions occurring in nature or with different chemical reactions.

Chemical materials may be any inorganic or organic chemical material obtained by reacting inorganic and/or organic chemical reactants. The inorganic and organic chemical reactants may be natural chemical materials or may be synthetic chemical materials. Chemical materials may include one or more polymers. Chemical materials may include one or more chemical compounds. In an embodiment, chemical material may refer to an organic chemical compound. The chemical material may refer to one or more polymer(s) and/or one or more functional chemical compound(s). Chemical reactions may include any chemical reaction commonly known in the state of the art in which the reactants are converted to one or more different chemical materials. Chemical reactions may involve the use of catalysts, enzymes, bacteria, etc. to achieve the chemical reaction between the reactants. The chemical material may be characterized by at least one functional group. The functioncal group may be at least one of alkyl group, alkenyl group, alkynyl group, phenyl group, carbonyl group, ketone group, aldehyde group, hydroxyl group, haloformyl group, ester group, carboxylate group, halo group, carboxyl group, peroxy group, carboalkoxy group, hydroperoxyl group, ether group, acetal group, hemiacetcal group, hemiketal group, ketal group, carboxylic anhydride group, carboxamide group, amidine group, amine group, ketamine group, aldimine group, imide group, cyante group, azo group, nitrite group, nitrate group nitro group, nitrile group, sulfide group, thiol group, sulfinyl group, sulfonyl group, sulfo group, thiocyanate group, thionoester group, thiolester group, phosphino group, phosphono group, phosphate group or any combination thereof.

The biodegradability measure may relate to a quantity characterizing the time evolution of the biodegradation process. The biodegradability property may relate to a measurement quantity characterizing the time evolution of the biodegradation process. The biodegradability property may include a reference quantity as measured under reference measurement conditions. Reference quantities and reference measurement conditions may be provided by OECD, ASTM, ISO Standards or other applicable Standards such as the ones cited below.

To evaluate biodegradation currently a series of standardized tests, are used. For biodegradability, a variety of tests exists with specified conditions (e.g. ISO13432; December 2000, ISO14852; October 2004, ISO14855; April 2013, ISO17556; December 2012 and OECD 301; July 1992). Standardized tests often strike a balance between a time-efficient testing (from 14 days, up to 24 months) and real-life conditions. Companies developing new chemical materials need to invest significant resources in self-assessing product sustainability and in certification. The overall biodegradability assessment, including laboratory spaces and equipment, becomes costly and time consuming. The methods disclosed herein enable early identification the biodegradability of a new chemical material, already in the development process. The proposed method of measuring biodegradability as disclosed herein enables a fast and efficient way of developing new chemical materials. In an early phase, even before synthesis of the chemical material, the biodegradability can be measured. This allows to determine whether the chemical product is suited for market entry. This leads to a faster time to market. This also allows to reduce waste production, because the chemical material does not need to be synthesized to determine biodegradability.

Biodegradable substances or materials may be designed to degrade upon disposal by the action of living organisms. Biodegradability may relate to the environmental fate and/or behavior of the substance. Biodegradability may relate to the extent to which the substance can be decomposed by microorganisms such as such as—including but not limited to—enzymes, bacteria, fungi and/or algae. Biodegradability may be dependent on the substance's or material's chemical structure, chemical weight, physical factors such as cross-linking density, branching, crystallinity or solubility, and exposure conditions such as habitat like soil, compost or aquatic system. With respect to exposure conditions the microorganisms, microbial population, nutrient concentration, temperature, pH, pO2, ionic condition, or substrate characteristics such as toxicity may influence biodegradability. Biodegradability may be measured based on measured—butis not limited to—mass loss (mg/time), dissolved organic carbon (DOC, organic carbon concentration/time), oxygen consumption (e.g. though pressure measurement, e.g. Pa/time) or carbon dioxide production over time (e.g. though pressure measurement, e.g. Pa/time).

To quantify biodegradability in the sense of a measured property of the substance or chemical material is challenging and many measurement standards have been developed. Different measurement methods are defined to determine biodegradability under pre-defined laboratory conditions. For example, for wastewater OECD Test No. 301: “Ready Biodegradability” (Jul. 17, 1992) describes 6 methods for determination of biodegradability. Further for example, ASTM D5988-18 “standard test method for determining aerobic biodegradation of plastic materials in soil” describes the measuring of the carbon dioxide developed by microorganisms as a function of time of exposure, thus measuring the degree of biodegradability relative to a reference material. Further for example ISO 17556:2019 “plastics-determination of the ultimate aerobic biodegradability of plastic materials in soil by monitoring the oxygen demand in a respirometer or the amount of carbon dioxide evolved” yields the optimum rate of biodegradation of plastic material in a test soil by controlling the oxygen consumption or the carbon dioxide production. Further for example, ISO 14855-1:2012 “determination of the ultimate aerobic biodegradability of plastic materials under controlled composting conditions-method by analysis of evolved carbon dioxide-Part 1: General method” and ASTM D5338-15 “standard test method for determining aerobic biodegradation of plastic materials under controlled composting conditions, incorporating thermophilic temperatures” determine the ultimate aerobic biodegradability (means by which microorganisms entirely consume a chemical or organic substance in the presence of oxygen) of plastics based on organic compounds under controlled composting conditions by measuring the percentage conversion of the carbon into carbon dioxide and the degree of disintegration of the plastic at the end of the test. ASTM D6400-21 “standard specification for labeling of plastics designed to be aerobically composted in municipal or industrial facilities” additionally includes elemental analysis, plant germination (phytotoxicity), and mesh filtration of the resulting particles. In ISO 17088:2021 “plastics-organic recycling-specifications for compostable plastics” includes the evaluation of negative consequences on the composting process and facility and negative effects on the quality of the resulting compost, including the presence of high levels of regulated metals and other harmful components.

For aerobic biodegradation ISO 18830:2016 “plastics—determination of aerobic biodegradation of non-floating plastic materials in a seawater/sandy sediment interface—method by measuring the oxygen demand in closed respirometer”, ISO 19679:2020 “plastics—determination of aerobic biodegradation of non-floating plastic materials in a seawater/sediment interface—method by analysis of evolved carbon dioxide” were developed. The biodegradation evaluation is measured by the oxygen demand or the CO2 evolution. Further standards for example include ISO 14853:2016 “plastics—determination of the ultimate anaerobic biodegradation of plastic materials in an aqueous system—method by measurement of biogas production”, ISO 23977-1:2020 “plastics—determination of the aerobic biodegradation of plastic materials exposed to seawater—Part 1: method by analysis of evolved carbon dioxide” and ISO 23977-2:2020 “plastics—determination of the aerobic biodegradation of plastic materials exposed to seawater—Part 2: method by measuring the oxygen demand in closed respirometer”.

The quantified biodegradation property or measure for the chemical material or substance may depend on the measurement method and conditions used, the measurement environment and the measurement value related to the degradation process, such as—but not limited to—mass loss, DOC, oxygen consumption or carbon dioxide production over time. The measurement method and the measured characteristics may be provided as metadata per measurement point related to biodegradability.

The digital representation associated with the biodegradation habitat may include a microbial composition of the biodegradation habitat, e.g. including at least one microbial community, including an enzyme environment including one or more enzyme(s), one or more bacteria one or more algae and/or one or more fungi. The digital representation associated with the biodegradation habitat may include exposure conditions such as—but not limited to—the microorganisms, microbial population, nutrient concentration, temperature, pH, pO2, ionic condition, or substrate characteristics such as toxicity that influence biodegradation process. The digital representation associated with the biodegradation habitat may relate to any one or more of a marine habitat, a waste water habitat, a limnic habitat, a compost habitat, an anaerobic habitat or a soil habitat.

The biodegradation mechanism or process may include one or more steps based on microbiological processes for degradation of the chemical material. The biodegradation mechanisms may include multiple steps including one or more fragmentation stages.

As an example, degradation, according to literature such as Dussud C., Ghiglione J. F. Bacterial degradation of synthetic plastics. [(accessed on 5 Apr. 2024)]; CIESM Workshop Monogr. 2014 46:49-54. Available online: https://oceans.taraexpeditions.org/en/m/science/news/bacterial-degradation-of-synthetic-plastics [Google Scholar] may include:

Bio-deterioration or biofilm formation, wherein microbial metabolic activity may result in plastic cracks affecting the physical properties or changes in the microstructure of the matrix by pH change as a result of the released acid or biofilm formation.

Bio-fragmentation of the chemical material, such as polymer chains, wherein the activity of enzymes produced by microorganisms may lead to sub-group splitting such as oligomer and/or polymer sub-groups.

Degradation of sub-groups oligomers and/or monomers, wherein sub-groups oligomers and/or monomers enter inside the cells, and secondary degraders assimilate sub-groups oligomers and/or monomers as a carbon source, thus increasing the microbial biomass.

Assimilation of sub-groups oligomers and/or monomers and excretion of completely oxidized metabolites to H2O, CO2, N2, and CH4.

The break down model may be or include an enzymatic pathway model that maps the chemical material in relation to the degradation process related to enzyme(s). The break down model may include a database providing enzymatic pathways for chemical material(s) and/or one or more sub-groups of the chemical material. The break down model may be configured to search the chemical material provided by the digital representation of the chemical material to enzyme(s) provided by the representation of the habitat to enzymatic pathways. Examples of enzymatic pathway models are disclosed in literature such as Sara Calhoun, Magdalena Korczynska, Daniel J Wichelecki, Brian San Francisco, Suwen Zhao, Dmitry A Rodionov, Matthew W Vetting, Nawar F Al-Obaidi, Henry Lin, Matthew J O'Meara, David A Scott, John H Morris, Daniel Russel, Steven C Almo, Andrei L Osterman, John A Gerlt, Matthew P Jacobson, Brian K Shoichet, Andrej Sali (2018) Prediction of enzymatic pathways by integrative pathway mapping eLife 7:e31097.

The breakdown model may be trained based on historic measurement data relating to the biodegradation process and/or the habitat the process takes place in. The training data may include the representation of the chemical material, the representation of the habitat the chemical material was exposed to and one or more fragment(s) of the chemical material measured at one or more time intervals or time steps. In particular, the training data may include the representation of the chemical material, the representation of the habitat the chemical material was exposed to and one or more fragment(s) of the chemical material measured at a defined time step of a measurement process such as the end of the biodegradation process or at the end-of-life of the material. The end of the process may relate to the time step or interval defined according to measurement standards e.g. as described above or to the time step after which the change of fragments measured is below a defined threshold.

One or more representation(s) of one or more break down product(s) or fragment(s) present in one or more sample(s) of the chemical material exposed to the biodegradation habitat for one or more defined time interval(s) or time steps may be provided. The fragment(s) of the biodegradation process may be monitored based on the fragmentation stages. For example, one or more samples of the chemical material exposed to the biodegradation habitat for one or more defined time interval(s) or time steps may be measured with respect to the fragments that have formed up to the certain time interval(s), such as—but not limited to—after 28 days of exposure. The composition of fragments present in the one or more samples of the chemical material exposed to the biodegradation habitat may be provided per time interval or time step, e.g. as measured by standard methods such as NMR, GC or Mass Spec.

The breakdown model may relate to a (retro-)synthesis model mapping target chemical materials or organic molecules to precursors that may form the chemical material or target molecule through reaction pathways. The breakdown model may relate to one or more defined time interval(s) or time point(s) of biodegradation process. The breakdown model may relate to one or more habitat representation(s), e.g. per habitat representation. The breakdown model may relate to an at least partially data-driven model. Examples of models may include retrosynthesis models as for example described in Yijia Sun, Nikolaos V Sahinidis, Computer-aided retrosynthetic design: fundamentals, tools, and outlook, Current Opinion in Chemical Engineering, Volume 35, 2022, 100721, ISSN 2211-3398, https://doi.org/10.1016/j.coche.2021.100721, https://www.sciencedirect.com/science/article/pii/S2211339821000538. Examples of models may include (retro-)synthesis models as for example described in Connor W. Coley, William H. Green, and Klavs F. Jensen, Machine Learning in Computer-Aided Synthesis Planning, Accounts of Chemical Research 2018 51 (5), 1281-1289, DOI: 10.1021/acs.accounts.8b00087. Examples of models may include (retro-)synthesis models as for example described in Thomas J. Struble, Juan C. Alvarez, Scott P. Brown, Milan Chytil, Justin Cisar, Renee L. DesJarlais, Ola Engkvist, Scott A. Frank, Daniel R. Greve, Daniel J. Griffin, Xinjun Hou, Jeffrey W. Johannes, Constantine Kreatsoulas, Brian Lahue, Miriam Mathea, Georg Mogk, Christos A. Nicolaou, Andrew D. Palmer, Daniel J. Price, Richard I. Robinson, Sebastian Salentin, Li Xing, Tommi Jaakkola, William. H. Green, Regina Barzilay, Connor W. Coley, and Klavs F. Jensen, Current and Future Roles of Artificial Intelligence in Medicinal Chemistry Synthesis, Journal of Medicinal Chemistry 2020 63 (16), 8667-8682, DOI: 10.1021/acs.jmedchem.9b02120.

The breakdown model may include a pre-trained data driven model. The breakdown model may be trained on training data relating to one or more defined time interval(s) or time point(s) of biodegradation process. The breakdown model may be trained on training data relating to one or more habitat representation(s), e.g. per habitat representation. The pre-trained data driven model may be trained on historical chemical material data associated with the chemical material and respective or corresponding precursor data associated with the precursors that form the chemical material. The pre-trained model may be further trained based on the historic measurement data relating to the biodegradation process e.g. as described above.

The breakdown model may be configured to map the representation of the biodegradable chemical material e.g. depending on the representation of the biodegradation habitat to the break down product(s) or fragment(s) the chemical material degrades to. The breakdown model may be configured to map depending on the model for one or more defined time interval(s) or time point(s) and/or on one or more representations of the biodegradation habitat(s). The trained breakdown model may be selected based on respective one or more defined time interval(s) or time point(s) and/or on one or more representations of the biodegradation habitat(s) the model is trained on.

At least one ecotoxicological measure may be determined based on the generated break down product(s) as provided by the breakdown model(s). The ecotox model or mapping may be based on providing the at least one ecotoxicological measure by a data base storing ecotoxicological measures in dependence on the molecular structure(s).

The biodegradability of the chemical material may be determined based on the representation of the chemical material and/or the representation of the habitat. The digital representation of the chemical material associated with physicochemical characteristics of the chemical material may be provided. A biodegradation habitat may be provided, wherein a biodegradation habitat may be related to habitat descriptor values of habitat descriptors influencing a biodegradation of the chemical material in the respective habitat, wherein the habitat descriptors are indicative of environmental characteristics of the habitat. A biodegradation model based on the provided biodegradation habitat may be provided. The biodegradation model may be adapted to determine a biodegradability of the chemical material in the respective biodegradation habitat. The biodegradation model may be a data driven model parametrized with respect to the biodegradation habitat such that it can determine a biodegradability of a chemical material based on the physicochemical characteristics. The biodegradability of the chemical material may be provided based on the provided biodegradation model and the digital representation of the chemical material. The biodegradability may be determined as described in WO2023156616A1, the full disclosure of which is herewith incorporated by reference.

Microplastics are a major concern in development of chemical products. Microplastics may be avoided if the chemical product is fully biodegradable, meaning that the remaining products of the biodegradation process are CO2, minerals or biomass. The proposed method of measuring biodegradability as disclosed herein enables a fast and efficient way of developing new chemical materials that are fully biodegradable.

In particular in personal care products, it is desirable to develop soluble chemical materials that are fully biodegradable in an aqueous habitat. Another important target of the chemical industry is the need for biodegradable chemical products, to improve the sustainability of detergent formulations and to avoid the accumulation of non-biodegradable polymers in the ecosystem.

In the following, embodiments of the present disclosure will be outlined by ways of examples. It is to be understood that the present disclosure is not limited to said embodiments and/or examples.

In an embodiment, chemical material may refer to an organic chemical compound. The chemical material may refer to polymer(s) and/or functional chemical compound(s).

In an embodiment Polymer may refer to a synthetic polymer. In an embodiment, the synthetic polymer may be a chemical compound which is produced by a chemical production from one or more starting material(s), such as monomers, and which comprises at least two monomer units. The monomer units may be regarded as subunits of the synthetic polymer. The synthetic polymer may be prepared from the monomers by commonly known polymerization reactions. The synthetic polymer may be produced from a single type of monomers or from different monomers. The monomer units may be distributed randomly or may be present as blocks within the synthetic polymer. The synthetic polymer may be a linear polymer. The synthetic polymer may be a branched polymer. The synthetic polymer may be a cross-linked polymer. In an embodiment, synthetic polymer may refer to synthetic organic polymers. The synthetic organic polymer corresponds to one of the following classes: polyalkoxylate, polyester, polyamine, polyaminoester, polyamidoamine, polyurethane, polyol.

In an embodiment functional chemical compound may refer to molecules having a molecular mass below 10000 g/mol. More Optionally the chemical compound has a molecular weight of less than 600 g/mol, even more Optionally of less than 300 g/mol. Further, it is preferred that the functional chemical compound is present in the environment in a form that allows to completely describe the molecules using simple structural formulas, that contain the relevant information. A simple molecular structure refers to molecules that can be unambiguously described by covalent bindings between the atoms of the molecule. Examples, where this is not the case, are e.g. systems with dynamic equilibria between several forms like monomer and oligomers as in the case of several inorganic acids, or ionic species with very localized charge that strongly interacts with a solvent, e.g. via hydrogen bonding. In an embodiment, functional chemical compound may include one or more ingredients in a formulation.

The functional chemical compound may have at least one of the following properties: having an effect on a living organism's body, being suitable for influencing the structure or, being suitable for influencing the functioning of a living organism's body. In an embodiment, the functional chemical compound comprises at least one of the following functional groups: ester group, ether group, lactone group, hydroxyl group, carbonyl group, phenol group, amide group, amine group, alkyl group, alkylen group, phenyl group, ketone group, aldehyde group, acetal group, ketal group, sulfhydryl group, sulfide group or a combination thereof.

In an embodiment biodegradation habitat may refer to an environment in which biodegradation occurs. In an embodiment the biodegradation habitat may comprise a biological community, such as the presence of microorganisms and other organisms that facilitate the breakdown of the chemical material. The biodegradation habitat may refer to a property of the biodegradation habitat. The biodegradation habitat may refer to a property of the biodegradation habitat and an associated property value. In an embodiment, the biodegradation habitat may refer to one or more properties of the biodegradation habitat. In an embodiment, the biodegradation habitat may refer to one or more properties of the biodegradation habitat and respective associated one or more property values. In an embodiment, the biodegradation habitat property may refer to any one of a marine habitat, a waste water habitat, a fresh water habitat, a limnic habitat, an anaerobic habitat, a compost habitat or a soil habitat.

In an embodiment providing the chemical material may refer to providing a digital representation of the chemical material. Wherein the digital representation may be indicative or associated with physico-chemical properties of the chemical material, in particular the organic chemical compound, more particular the organic synthetic polymer and/or the functional chemical compound. The digital representation may include a unique identifier associated with the chemical material. In an embodiment, the digital representation may include a CAS number indicative of the chemical material. In another embodiment, the digital representation may include a IUPAC name indicative of the chemical material. In an embodiment, the digital representation may include a SMILES representation. In an embodiment, the digital representation may include a graph representation of the chemical material.

Providing a digital representation of an organic synthetic polymer may refer to physicochemical characteristics, for example, in form of values for respective quantities. However, the digital representation may also be a link to the respective physicochemical characteristics via which the physicochemical characteristics may be accessed, or the digital representation may refer to an identifier that is associated with the physicochemical characteristics and allows to utilize a respective look up storage in order to access the physicochemical characteristics. Moreover, the digital representation may also refer to information that allows to derive the physicochemical characteristics using one or more known relations. For example, a synthesis specification or a structural formula of a polymer may be utilized as digital representation. This may allow to derive, using known chemical and physical laws and relations, respective physicochemical characteristics.

The following table shows an example of a synthesis specification of a polymer in accordance with the disclosure.

| | | | | reaction | | | post |
|---|---|---|---|---|---|---|---|
| | | | | tempera- | dosing | pres- | reaction |
| starter | monomer 1 | monomer 2 | catalyst | ture | time | sure | time |
| [mol] | [mol] | [mol] | [mol %] | [°C] | [h] | [bar] | [h] |
| diethylene | ethylene | propylene | potassium | | | | |
| glycol | oxide | oxide | hydroxide | | | | |
| 1 | 32 | 0 | 0.03 | 140 | 15 | 4 | 3 |

Generally, throughout the following description referring a parameter or a characteristic comprises referring both to the respective quantity and also to a specific value of the quantity if not explicitly defined otherwise. For example, a parameter being a temperature always refers to the quantity being a temperature and also to a specific value of the temperature being set for the quantity. Since in most cases the explicit value of the parameter can be different for different embodiments and application cases the value is generally not mentioned. However, providing a parameter or characteristic generally means providing the quantity, e.g. the information that a value is a temperature, and also the value of the quantity or characteristic itself.

In particular, the physicochemical characteristics of a polymer can be quantified by polymer physicochemical parameters. Optionally, the digital representation is indicative of and/or comprises polymer physicochemical parameters, wherein the polymer physicochemical parameters are indicative of the physicochemical characteristics of the polymer. In particular, the polymer physicochemical parameters are indicative of parameters quantifying the physicochemical characteristics of the polymer. In this context, the term “physicochemical characteristics” may include or relate to physical and/or chemical characteristics of the polymer. However, the digital representation can also be provided such that it allows to derive physicochemical characteristics, for instance, by providing a representation of the polymer for which respective physicochemical characteristics are already stored or can be determined. The digital representation may relate to at least one of a synthesis specification, a structural formula, a brand name, an IUPAC name, a chemical identifier and a CAS number of the polymer.

In another embodiment, the polymer physicochemical parameters are parameters quantifying the physicochemical characteristics of subgroups of the polymer. In this embodiment, the digital representation can also be provided such that it allows to derive the polymer physicochemical parameters by determining subgroups of the polymer and to determine the polymer physicochemical parameters based on physicochemical characteristics of the determined subgroups. Generally, a subgroup refers to a part of the polymer, wherein all subgroups of a polymer together form the polymer. For example, a subgroup can refer to a part of the polymer, wherein the subgroups are linked together successively along a chain or network to form the polymer. The subgroups of the polymer may include or relate to repeating units that describe a part of the polymer which when repeated produces the complete polymer chain. However, in some cases, a subgroup can also refer to a single part of the polymer that is not repeated. The subgroups may comprise parts that are repeated, for example, a subgroup of a polymer can comprise a repeating core also present in other subgroups and further additional parts that are not present in other subgroups. The subgroups may relate to or include at least one of polymerized monomers or oligomer fragments. The subgroups may relate to or include polymerized monomers. In this context, polymerized monomers refer to monomers after their polymerization sometimes also called “mer unit” or “mer”. In particular, polymerized monomers do not refer to monomers, i.e. raw materials, as present in a reaction mixture before polymerization, but refer to repeating units derived from monomers that have been changed during or after the polymerization. Thus, subgroup parameters determined for polymerized monomers are different from subgroup parameters determined for unreacted monomers before polymerization. It has been found by the inventors that in particular the polymerized monomers allow to determine polymer parameters from the subgroup parameters of the polymerized monomers that allow for an accurate determination of the biodegradability. In another embodiment, the digital representation of the polymer comprises subgroups provided as molecular model which is indicative of its chemical structure of the subgroup after its polymerization. The molecular model of a subgroup may be determined in a way that is suited for quantum chemical computations regarding a number and type of atoms and their connectivity that is representative of the properties of the subgroup within the polymer. Moreover, additionally an alternatively to a molecular model of a subgroup treating the subgroup as a monomer structure, also a molecular model referring to an oligomer model can be utilized that takes into account effects of neighboring molecular structures of the subgroup in the polymer.

Generally, if the digital representation of the polymer does not directly comprise the polymer physicochemical parameters, it is preferred that the polymer physicochemical parameters are determined by determining the subgroups of the polymer. For example, respective subgroups of the polymer can be determined utilizing known methods. However, it is preferred that the determination of the subgroups of the polymer is performed in accordance with later described embodiments of the invention. In particular, it is preferred that the subgroups are determined such that between atoms of different subgroups in the polymer the bond is as least polarized as possible and, Optionally, with a bond order as small as possible (e.g. a CC single bond). Additionally, it is preferred that the subgroups representing a polymer comprise the same number of active non-hydrogenatoms then the polymer. Besides the active atoms, a subgroup can also contain further atoms, which can be ignored during computing the parameters of the subgroup. Further, it is preferred that the subgroups are determined in a way that polymers comprising parts, which were built up with different polymerization techniques, are well covered and fulfill the foresaid conditions. An example is a polyether used as ingredient for a polyurethane. Generally, a database or archive with a plurality of reactions between polymer parts can be generated and the subgroups can be derived from the respective structure of the reactions. For example, specific chemical languages like SMILES and SMARTS can be utilized to easily derive the subgroup of a polymer. For example, a database of reaction SMARTS can be generated and then based on the polymerization of the respective polymer a corresponding reaction SMARTS can be selected. From the selected reaction SMARTS then the SMILES of monomers of the polymer are directly derivable and, for example, RDkit can be used to determine from the SMILES of the monomers the SMILES, i.e. the number and connectivity of the atoms, of the subgroups.

The determined subgroups of the polymer are associated with subgroup physicochemical parameters quantifying physicochemical characteristics of the subgroups in the polymer, Optionally, also the subgroup physicochemical parameters refer to subgroup parameters. In particular, it is preferred that if the polymer physicochemical parameters are not directly provided by the digital representation, the polymer physicochemical parameters are determined by determining a respective subgroup physicochemical parameter for each of the subgroups and to determine the polymer physicochemical parameters based on the subgroup physicochemical parameters of the subgroups, for instance, by averaging. Thus, the method Optionally comprises first providing or determining for the polymer the subgroups from the digital representation of the polymer, then to determine or provide the subgroup physicochemical parameters, i.e. values of the parameters quantifying the physicochemical characteristics, of the subgroups, and then to determine the polymer physicochemical parameters based on the subgroup physicochemical parameters of each polymer.

Optionally, the polymer physicochemical parameters may refer to polymer parameters referring to at least one of constitutional parameters, count parameters, list of structural fragments, fingerprints, graph invariants, 3D-parameters and/or higher dimensional parameters that are indicative of parameters quantifying physicochemical characteristics of the polymer. In a preferred embodiment the polymer parameters refer to 3D parameters, in particular, quantum chemical parameters. Moreover, the inventors have found that in particular a molar mass describes the biodegradation of a polymer very accurately. Thus, it is in particular preferred that the physicochemical parameters comprise a molar mass of the polymer. Generally, the polymer physicochemical parameters can be derived from the subgroup physicochemical parameters, thus, also the subgroup physicochemical parameters can refer to the same parameters as stated above. However, the physicochemical parameters can also be derived without utilizing subgroups, for instance, by quantum chemical simulations of the whole polymer. In the following the possible physicochemical parameters are defined in more detail. Also in these cases the defined physicochemical parameters can refer directly to the polymer physicochemical parameters or, optionally, to the subgroup physicochemical parameters.

A constitutional parameter can refer to any of a potential, average molecular weight, polydispersity, charge, spin, boiling point, melting point, enthalpy of fusion, dissociation constant, Hansen parameter, protic, polar and dispersive contributions, Abraham parameter, retention index, TPSA, receptor binding constant, Michaelis-Menten constant, Inhibitor constant, Mutagenicity, LD50, bioconcentration, toxicity, biodegradation profile and viscosity.

A count parameter can refer to any of a sum of atomic electro negativities, a sum of atomic polarizabilities, an amount of ingredients, a ratio of amounts of ingredients, a number of atoms and non H-atoms, a number of H, B, C, N, O, P, S, Hal and heavy atoms, a number of H-donor and H-acceptor atoms, a number of bonds, non-H or multiple bonds, a number of double, triple and aromatic bonds, a number of functional groups, a ratio of functional groups, a sum of bond orders, an aromatic ratio, a number of rings or circuits, a number of unpaired electrons, a number of rotatable bonds, rotatable bond fractions, and a number of conformers.

Polymer parameters referring to a list of structural fragment parameters can refer to at least one of a list of molecular fractions, a list of functional groups, a list of bonds, and a list of atoms. Fingerprint parameters comprise Optionally, at least one of MACCS keys, Optionally, in bit format or total amount format, Morgan and other circular fingerprints, Optionally, in bit format or total amount format, topological torsion, atom pairs, infrared and related spectra, fingerprint count, PubChem fingerprint, substructure fingerprint, and Klekota-Roth fingerprint. Graph invariants/topological indices parameters comprise Optionally at least one of topostructural indices and topochemical indices.

In a preferred embodiment the polymer physicochemical parameters are 3D parameters comprisesing at least one of a volume as sum overall atoms, a mean volume per atom, an area as sum overall atoms, an area as mean per atom, an area over all atoms, an area as mean per atom, a solvent accessible surface, a dispersion energy, a dielectric energy, a H-donor, H-acceptor, polar and non-polar surface area, an atom resolved H-donor, H-acceptor, polar and non-polar surface area, a shape, a sphericity, dipole and higher electric moments, polarizability, dielectric energy, protic, polar and non-polar surface area, orbital energies and orbital gaps, ionization energy, electron affinity, hardness, electronegativity, electrophilicity, excitation energies and intensities, infrared and ultraviolet absorption bands, reactivity measurements, redox potential, bond criterial points, partial charges, charge surface areas, atomic orbital contributions, bond orders, atom radius. In particular, it is preferred that the polymer physicochemical parameters refer to 3D parameters comprising at least one of a sum of a volume over all atoms, a mean of a volume per atom, a sum of the area over all atoms, a mean of an area per atom, a solvent accessible surface, a dispersion energy, a dielectric energy, a H-donor, H-acceptor, polar and/or non-polar surface area, atom resolved H-donor, H-acceptor, polar and/or non-polar surface area, shape, sphericity, cone angles, polarizability, dielectric energy, protic, polar and/or non-polar surface area, excitation energies and intensities, infrared and/or UV absorption bands, reactivity measurements, particle charges and/or charge surface areas. On utilized higher dimensional parameter can comprise at least one of a conformational partition function, solubility, vapor pressure, activity coefficient, diffusion coefficient, partition coefficient, interfacial activity, rotational constant, moment of inertia, radius of gyration, compositional drift of polymer, density, viscosity, conformer weighted volume and area, conformer weighted H-donor, H-acceptor, protic, polar and/or non-polar surface area, charge distribution, conformational dipole moment and molecular refraction. Optionally higher dimensional parameters are utilized that comprise at least one of solubilities, vapor pressure and activity coefficients, interfacial activity, conformer weighted H-donor, H-acceptor, protic, polar and non-polar surface area, and charge distribution.

In an embodiment, providing a digital representation of a functional chemical compound may refer to, a chemical structure of the functional chemical compound. As described above, the functional chemical compound can also comprise more than on chemical structure. In this case it is preferred that the digital representation provides a chemical structure that is associated and/or indicative of one or more structural formula(s) and a quantity ratio of the more than one structural formulas present in the functional chemical compound. The at least two structural formulas may be provided for functional chemical compound by the digital representation correspond to structural formulas related via chemical equilibrium.

Since the structural formulas of a functional chemical compound can be influenced by the habitat, it is preferred that the chemical structure provided by the digital representation depends on the habitat. For example, in aqueous media a certain molecule may tend to be present in a protonated form with a ratio unprotonated to protonated of 1:3. A digital representation of such a molecule by one chemical structure may in this case not be sufficient. Such a molecule may be represented by a digital representation comprising a quantity ratio indicative of the equilibrium between different structures associated with one chemical formula, wherein the more than one structure are in a chemical equilibrium. The digital representation may be referred to as a statistical representation due to the incorporation of the statistical frequency of molecules associated with each structure. To outline the concept the equilibrium as example sulfuric acid and one of its deprotonated structures is illustrated by H2SO4 (25%)⇔H++HSO4- (75%). Monohydrogensulfate and Dihydrogensulfate may both be a result of introducing Dihydrogensulfate into water with a tendency towards monohydrogensulfate, for example, the ratio may be 1:3 Dihydrogensulfate to Monohydrogensulfate. Hence, the digital representation of introducing Dihydrogensulfate into water may refer to a specification of the structure of Monohydrogensulfate and Dihydrogensulfate with respective quantities or a ratio of quantities. An example for a specification of a chemical structure may be the number and type of atoms and their respective connectivity. Another example comprises using SMILE and/or SMARTS for representing the chemical structure of a functional chemical compound.

Optionally, the digital representation comprises as characterizing parameters physicochemical characteristics of the functional chemical compound. In particular, the physicochemical characteristics of a functional chemical compound can be quantified by physicochemical parameters. Optionally, the digital representation may be indicative of and/or comprises physicochemical parameters, Optionally, referring to respective parameters, wherein the physicochemical parameters are indicative of the physicochemical characteristics of the functional chemical compound. In particular, the physicochemical parameters are indicative of parameters quantifying the physicochemical characteristics of the functional chemical compound. In this context, the term “physicochemical characteristics” refers to physical and/or chemical characteristics of the functional chemical compound. However, the digital representation can also be provided such that it allows to derive physicochemical characteristics, for example, for instance, by providing a representation of the functional chemical compound for which respective physicochemical characteristics are already stored or can be determined, for instance, by respective calculations. Optionally, the digital representation refers to at least one of a recipe, a structural formula, a brand name, an IUPAC name, a chemical identifier and a CAS number of the functional chemical compound.

Optionally, the physicochemical parameters refer to at least one of constitutional parameters, count parameters, list of structural fragments, fingerprints, graph invariants, 3D-parameters and/or higher dimensional parameters that are indicative of parameters quantifying physicochemical characteristics of the functional chemical compound. In a preferred embodiment the functional chemical compound parameters refer to 3D parameters, in particular, quantum chemical parameters. Moreover, the inventors have found that in particular a molar mass describes the biodegradation of a functional chemical compound very accurately. Thus, it is in particular preferred that the physicochemical parameters comprise a molar mass of the functional chemical compound. In the following the possible physicochemical parameters are defined in more detail.

A constitutional parameter can refer to any of a potential, average molecular weight, polydispersity, charge, spin, boiling point, melting point, enthalpy of fusion, dissociation constant, Hansen parameter, protic, polar and dispersive contributions, Abraham parameter, retention index, TPSA, receptor binding constant, Michaelis-Menten constant, Inhibitor constant, Mutagenicity, LD50, bioconcentration, toxicity, biodegradation profile and viscosity.

A count parameter can refer to any of a sum of atomic electro negativities, a sum of atomic polarizabilities, an amount of ingredients, a ratio of amounts of ingredients, a number of atoms and non H-atoms, a number of H, B, C, N, O, P, S, Hal and heavy atoms, a number of H-donor and H-acceptor atoms, a number of bonds, non-H or multiple bonds, a number of double, triple and aromatic bonds, a number of functional groups, a ratio of functional groups, a sum of bond orders, an aromatic ratio, a number of rings or circuits, a number of unpaired electrons, a number of rotatable bonds, rotatable bond fractions, and a number of conformers.

Parameters referring to a list of structural fragment parameters can refer to at least one of a list of molecular fractions, a list of functional groups, a list of bonds, and a list of atoms. Fingerprint parameters comprise Optionally, at least one of MACCS keys, Optionally, in bit format or total amount format, Morgan and other circular fingerprints, Optionally, in bit format or total amount format, topological torsion, atom pairs, infrared and related spectra, fingerprint count, PubChem fingerprint, substructure fingerprint, and Klekota-Roth fingerprint. Graph invariants/topological indices parameters comprise Optionally at least one of topostructural indices and topochemical indices.

In a preferred embodiment the functional chemical compound physicochemical parameters are 3D parameters comprising at least one of a volume as sum overall atoms, a mean volume per atom, an area as sum overall atoms, an area as mean per atom, an area over all atoms, an area as mean per atom, a solvent accessible surface, a dispersion energy, a dielectric energy, a H-donor, H-acceptor, polar and non-polar surface area, an atom resolved H-donor, H-acceptor, polar and non-polar surface area, a shape, a sphericity, dipole and higher electric moments, polarizability, dielectric energy, protic, polar and non-polar surface area, orbital energies and orbital gaps, ionization energy, electron affinity, hardness, electronegativity, electrophilicity, excitation energies and intensities, infrared and ultraviolet absorption bands, reactivity measurements, redox potential, bond criterial points, partial charges, charge surface areas, atomic orbital contributions, bond orders, atom radius. In particular, it is preferred that the functional chemical compound physicochemical parameters refer to 3D parameters comprising at least one of a sum of a volume over all atoms, a mean of a volume per atom, a sum of the area over all atoms, a mean of an area per atom, a solvent accessible surface, a dispersion energy, a dielectric energy, a H-donor, H-acceptor, polar and/or non-polar surface area, atom resolved H-donor, H-acceptor, polar and/or non-polar surface area, shape, sphericity, cone angles, polarizability, dielectric energy, protic, polar and/or non-polar surface area, excitation energies and intensities, infrared and/or UV absorption bands, reactivity measurements, particle charges and/or charge surface areas. A Optionally utilized higher dimensional parameter can comprise at least one of a conformational partition function, solubility, vapor pressure, activity coefficient, diffusion coefficient, partition coefficient, interfacial activity, rotational constant, moment of inertia, radius of gyration, compositional drift of functional chemical compound, density, viscosity, conformer weighted volume and area, conformer weighted H-donor, H-acceptor, protic, polar and/or non-polar surface area, charge distribution, conformational dipole moment and molecular refraction. Optionally higher dimensional parameters are utilized that comprise at least one of solubilities, vapor pressure and activity coefficients, interfacial activity, conformer weighted H-donor, H-acceptor, protic, polar and non-polar surface area, and charge distribution.

In an embodiment, providing the biodegradation habitat may refer to providing a digital representation of the biodegradation habitat. The digital representation of the biodegradation habitat may refer to a property of the biodegradation habitat. The digital representation of the biodegradation habitat may refer to a property of the biodegradation habitat and an associated property value. In an embodiment, the digital representation of the biodegradation habitat may refer to one or more properties of the biodegradation habitat. In an embodiment, the digital representation of the biodegradation habitat may refer to one or more properties of the biodegradation habitat and respective associated one or more property values. The habitat properties may be indicative of environment characteristics of the habitat.

In particular, the environmental characteristics of a biodegradation habitat may influence a biological activity in the respective habitat, for example, the environmental characteristics may influence a presence, growth or absence of specific microorganisms. Thus, the environmental characteristics may be defined by the biodegradation habitat properties and may indirectly also influence the biodegradation of a chemical material in the respective habitat. For example, if a chemical material is biodegradable by a specific microorganism that needs a specific salt concentration, the chemical material will biodegrade fast in a habitat providing such a salt concentration, like a marine habitat, but will biodegrade much slower in a habitat with not the right salt concentration, like waste water. Again, being indicative of or associated with biodegradation habitat properties of a biodegradation habitat is defined as allowing to access the information of the biodegradation habitat properties. For example, the habitat may directly comprise the biodegradation habitat properties, for example, in form of values for respective quantities. However, the habitat can also be a link to the respective biodegradation habitat properties via which the biodegradation habitat properties may be accessed, or the habitat can refer to an identifier that is associated with the biodegradation habitat properties and allows to utilize a respective look up storage in order to access the biodegradation habitat properties. Moreover, the habitat may also refer to information that allows to derive the biodegradation habitat properties using one or more known relations. For example, a geolocation of an environment can be utilized together with the habitat allowing to derive, using knowledge on a respective geolocation, to derive respective biodegradation habitat properties

Optionally, the biodegradation habitat refers to any one of a marine habitat, a waste water habitat, a limnic habitat, an anaerobic habitat, a compost habitat or a soil habitat. In a preferred embodiment, the biodegradation habitat refers to a marine habitat and wherein the biodegradation habitat properties refer to at least one of a salt concentration, a sedimentation type, oxygen level, location, sample depth, a water temperature, a nutrient concentration, for example, a nitrogen, phosphate, potassium, and/or dissolved organic carbon concentration, a pH value, an environmental type, oxygen content, and a microbial community. In a further preferred embodiment, the biodegradation habitat refers to a limnic habitat and wherein the biodegradation habitat properties refer to at least one of a salt concentration, a sedimentation type, oxygen level, location, sample depth a water temperature, a nutrient concentration, a pH value, an environmental type and a microbial community. In a further preferred embodiment, the biodegradation habitat refers to waste water and the biodegradation habitat properties refer to at least one of a water temperature, a microbial community, a sludge concentration, a nutrient concentration, a pH value, a test duration, a solid content, and an enzyme environment. Moreover, in this habitat the sludge can also be a separate habitat. Thus, in an embodiment the habitat can also be a sludge habitat, for example, as the aerobic part of a waste water treatment plant and the biodegradation habitat properties refer to at least one of a solid content, pH, nutrient content, heavy metal content, microbial community. In a further preferred embodiment, the biodegradation habitat refers to soil and the biodegradation habitat properties refer to at least one of a temperature, composition, for example, a sand and/or clay content, a pH value, a moisture content, a nutrient concentration, a microbial community, a nitrogen content, a water holding capacity and an enzyme environment. In a further preferred embodiment, the biodegradation habitat refers to compost and the biodegradation habitat properties refer to at least one of a temperature, compost activity, a pH value, a moisture content, humidity, compost maturity, compost composition, compost origin, a nutrient concentration, a microbial community, a solid content, a water holding capacity, and an enzyme environment. Generally, the habitat can also refer to a habitat of a standard test utilized for determining biodegradability of a chemical material. For example, standard tests as defined by ISO13432, ISO14852, ISO14855, ISO17556 and OECD 301 also define a specific habitat in which the biodegradation takes place. Thus the providing of the biodegradation habitat can also comprise providing, for instance, selecting via a user input, one of the standard tests, wherein the biodegradation habitat properties then refer to the specific characteristics of the test, i.e. of the test environment and thus test habitat. Moreover, the habitat can also be defined by the biodegradation of a reference chemical material or other reference chemical. In this case the habitat can be provided by providing the reference and its biodegradation. In this case the reference and its biodegradation are indicative of the biodegradation habitat properties. The microbial community may refer to microbes and/or bacteria and/or fungi. The microbial community may be indicative of an enzyme environment.

The method further comprises providing a biodegradation habitat, wherein a biodegradation habitat is indicative of habitat biodegradation habitat property values of biodegradation habitat properties influencing a biodegradation of a chemical material in the respective habitat. In particular, the providing can refer to receiving the biodegradation habitat from an input of a user using, for instance, a respective input unit. Moreover, the providing can also refer to accessing a storage unit on which the biodegradation habitat is already stored. Furthermore, the providing can also refer to a presetting of a biodegradability habitat. For example, if the method is utilized in a very specific context that is only sensible with one specific biodegradation habitat, the respective biodegradation habitat can be preset and thus has not to be provided as specific input. Further, the providing can also comprise receiving directly the habitat biodegradation habitat property values of the biodegradation habitat properties, for instance, via a network connection, from other sources and providing the received habitat biodegradation habitat property values of biodegradation habitat properties as biodegradation habitat. The provided biodegradation habitat can refer to a general habitat, for instance, can refer to a waste water habitat, wherein respective habitat biodegradation habitat property values for the biodegradation habitat properties for this habitat are then already stored on a respective storage which can be accessed. However, the provided biodegradation habitat can also directly comprise the respective habitat biodegradation habitat property values for the biodegradation habitat to provide a further specification of the biodegradation habitat, for example marine benthic. Moreover, the providing of a biodegradation habitat can include providing a digital representation of the biodegradation habitat, wherein the digital representation can then be indicative of respective habitat biodegradation habitat property values of biodegradation habitat properties influencing a biodegradation of a chemical material in the respective habitat.

In an embodiment, breakdown product may refer to a portion of a chemical material after enzymatic induced degeneration of the chemical product. In an embodiment, initial breakdown product may refer to a breakdown product after a first step of enzymatic induced degeneration of the chemical material. In an embodiment residual breakdown product may refer to a breakdown product, that is (substantially) inert to enzymatic induced degradation of the chemical product in the biodegradation habitat. In an embodiment generating breakdown products may refer to determining breakdown products. In an embodiment, providing the breakdown products may refer to providing a digital representation of the breakdown products.

In an embodiment breakdown model may refer to a model relating a chemical material and the biodegradation habitat to breakdown products. In particular, the biodegradation habitat may be indicative of an enzyme environment in the biodegradation habitat.

In an embodiment the breakdown model may be a data driven model. The term “data driven” is used here to emphasize that the model is mainly based on respective data input and not, for instance, on intuition, personal experience, knowledge or physico-chemical models.

Optionally, the breakdown model refers to a machine learning based model that is based on known machine learning algorithms, like neural networks, regression models, classification algorithms, etc. It has been found that for most applications in this context, in particular, regression models based on Linear Regression, Random Forests, Boosted Trees, Lasso, Ridge Regression and MARS algorithms are suitable, whereas for classification models, in particular, Random Forests, Logistic Regression and SVM algorithms are suitable. Optionally, the breakdown model is based on a neural network algorithms. Generally, the breakdown model is parameterized during a training process in which physicochemical characteristics are utilized together with corresponding biodegradation habitats, indicative of an enzyme environment in the biodegradation habitat. Based on such a training data set that is specific for a biodegradation habitat, the respective parameters of the data driven model can be determined utilizing known training methods such that the breakdown model is also able to determine a breakdown product of chemical materials that are not part of the training data set.

In an embodiment the training data set may be obtained by the following method:

A sample of a biodegradation habitat is provided with a chemical material. In an example, the biodegradation habitat may be waste water. Degradation of the polymer initiates growth of microbes that are fed by the biodegradation of the polymer. In an enrichment step, a part of the sample is re-fed with the polymer and the process is repeated a number of times. This leads to a further increase of the microbe population that biodegrade the polymer. Breakdown products may be identified using analytical techniques suitable for identifying molecular bonds and structures (e.g. NMR, FTIR, GPC). The microbes may be analyzed using (DNA sequencing). From the microbes, enzymes relevant for biodegradation may be determined. By this training data may be generated that show a relation between the chemical product, biodegradation breakdown products and enzymes. In an alternative, training data may be generated that show a relation between the chemical product, biodegradation breakdown products, and microbes. In a further alternative, the training data may show a relation between breakable bonds in the chemical material, biodegradation breakdown products and microbes. In a further alternative, the training data may show a relation between breakable bonds in the chemical material, biodegradation breakdown products and enzymes. These relations may be trained into a breakdown model by the training methods described above. The biodegradation breakdown products of the polymer may be referred to as initial breakdown products.

The method for obtaining the training data may be applied repeatedly with the biodegradation breakdown products obtained in the previous step of biodegradation. The sample of the habitat is provided with the breakdown products obtained in the previous biodegradation step, in this example the initial breakdown products generated by the first degradation of the polymer. Degradation of the breakdown products obtained in the previous biodegradation step initiates growth of microbes that are fed by the biodegradation breakdown products obtained in the previous biodegradation step. In an enrichment step, the sample is refed with the breakdown products obtained in the previous biodegradation step. This leads to a further increase of the microbe population that biodegrade the obtained in the previous biodegradation step. Subsequent breakdown products may be identified using analytical techniques suitable for identifying molecular bonds and structures (e.g. NMR, FTIR, GPC). The microbes may be analyzed using (DNA sequencing). From the microbes, enzymes relevant for biodegradation may be determined. By this training data may be generated that show a relation between the breakdown products obtained in the previous biodegradation step, subsequent biodegradation breakdown products and enzymes. In an alternative, training data may be generated that show a relation between the breakdown products obtained in the previous biodegradation step, subsequent biodegradation breakdown products, and microbes. In a further alternative, the training data may show a relation between breakable bonds in the breakdown products obtained in the previous biodegradation step, subsequent biodegradation breakdown products and microbes. In a further alternative, the training data may show a relation between breakable bonds in the breakdown products obtained in the previous biodegradation step, subsequent biodegradation breakdown products and enzymes. These relations may be trained into a breakdown model by the training methods described above. These steps may be repeated until no further biodegradation is observed. By this, biodegradation breakdown products that can not be biodegraded further in the habitat may be identified. These may be referred to as inert breakdown products.

In an embodiment the breakdown model may be an enzymatic pathway model. The enzymatic pathway model may be rule based. Each reaction rule may be based on the ability of enzymes to catabolize the chemical material and subsequent biodegradation breakdown products. In an embodiment the model may further comprise a database linking enzymes to microbes producing these enzymes. In an embodiment the enzymatic pathway model may comprise a database linking biodegradation habitats to microbes in the biodegradation habitat, wherein the microbes produce enzymes. In an embodiment, the enzymatic pathway model may comprise a database linking biodegradation habitats to enzymes.

Fully biodegradable may refer to a chemical material that may be completely mineralized into biomass, H2O and CO2 under aerobic conditions or biomass, H2O, CO2 and CH4 under anerobic conditions.

The method allows you to determine whether the chemical material is biodegradable in the habitat, based on the enzymes that are present in the habitat.

In an embodiment the method further comprises providing a biodegradation test method, wherein the provided biodegradation test method is indicative of a standardised biodegradation test method for determining experimentally a biodegradation of a chemical material, wherein the enzyme environment is selected based on the provided biodegradation test method.

In an embodiment further comprises, wherein the biodegradation habitat refers to any one of a marine habitat, a waste water habitat, a limnic habitat, a compost habitat, an anaerobic habitat or a soil habitat.

In an embodiment further comprises, wherein the biodegradation habitat refers to a marine habitat and wherein the habitat descriptors refer to at least one of a salt concentration, a sedimentation type, oxygen level, location, sample depth, a water temperature, a nutrient concentration, a pH value, an environmental type and the enzyme environment.

In an embodiment further comprises, wherein the biodegradation habitat refers to waste water and the habitat descriptors refer to at least one of a water temperature, a microbial community, a sludge concentration, a nutrient concentration, a pH value, a test duration and the enzyme environment.

In an embodiment further comprises, wherein the biodegradation habitat refers to soil and the habitat descriptors refer to at least one of a temperature, a sand content, a pH value, a moisture content, a nutrient concentration, a microbial community and the enzyme environment.

In an embodiment further comprises, wherein the biodegradation habitat refers to compost and the habitat descriptors refer to at least one of a temperature, compost activity, a pH value, a moisture content, humidity, compost maturity, compost composition, compost origin, a nutrient concentration, a microbial community and an enzyme environment.

In an embodiment further comprises, wherein habitat property values for the habitat descriptors are stored associated with respective geolocations, wherein the providing of a biodegradation habitat refers to providing a geolocation of the habitat and retrieving the habitat property values for the geolocation from storage.

In an embodiment, the breakdown model may be a data driven model.

Using a data driven model has the advantage that there is no need to fully understand the complex reactions leading to biodegradation. This saves time as understanding the complex reactions leading to biodegradation requires many experiments.

In an embodiment, the breakdown model may be an enzymatic pathway model.

Using an enzymatic pathway model allows to determine the full biodegradation path. Thereby enabling to easily determine if inert breakdown products will remain. Furthermore, all breakdown products in the biodegradation process may be determined. Which allows to asses toxicology for all breakdown products.

In an embodiment, providing the breakdown products may comprise providing initial breakdown products. The initial breakdown products may be indicative of a first biodegradation step. This allows use of simple models which are fast to develop, which do not require a lot of training data, therefore requiring less experiments. In case of an enzymatic pathway model, the determination is more accurate and faster in determining, as is does not require for determine the full pathway.

In an embodiment, subsequent breakdown products may be determined from a database. The database may comprise breakdown products and their further breakdown pathways. This allows a very fast determination of subsequent breakdown products.

In an embodiment, toxicology the chemical material may be determined based on breakdown products and an ecotox model. This allows to determine toxicology throughout the full biodegradation process.

### BRIEF DESCRIPTION OF THE DRAWINGS

In the following, the present disclosure is further described with reference to the enclosed figures. The same reference numbers in the drawings and this disclosure are intended to refer to the same or like elements, components, and/or parts.

FIG. 1 shows schematically and exemplarily an embodiment of a system comprising an apparatus for generating biodegradation breakdown products of a chemical material,

FIG. 2 shows schematically and exemplarily a flow chart of a method for generating breakdown products of a chemical material,

FIG. 3 shows schematically and exemplarily a flow chart of a method for training a biodegradation model for determining a biodegradability of a polymer,

FIG. 4 shows an exemplary system for producing a chemical product

FIG. 5 shows schematically and exemplarily an enzymatic pathway of biodegradation,

FIG. 6 shows schematically and exemplarily an output and input screen of an exemplary user interface.

The following embodiments are mere examples for implementing the method, the system or application device disclosed herein and shall not be considered limiting.

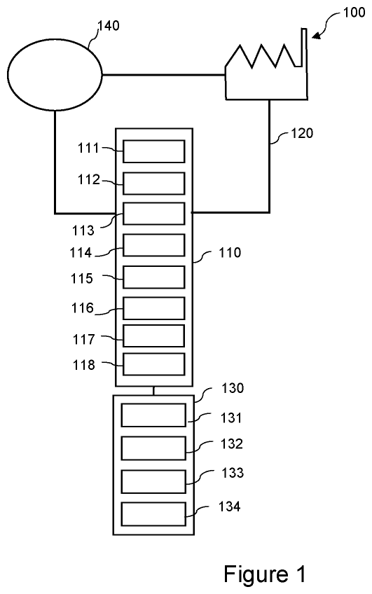

FIG. 1 shows schematically and exemplarily an embodiment of a system 100 comprising an apparatus 110 for generating breakdown products of a chemical material based on a digital representation of the chemical material and a provided biodegradation habitat, indicative of an enzyme environment in the biodegradation habitat. Further, the system 100 may comprise a training apparatus 130 for training a breakdown model utilized in the apparatus 110, a database 140 on which generating results of the breakdown products of chemical materials can be stored and a production system 120 for producing a product, in particular, comprising the chemical material, that can be controlled utilizing the generated breakdown products.

The apparatus 110 comprises a digital representation providing unit 111, a habitat providing unit 112, a model providing unit 113, a determination unit 114 and optionally an output and/or control unit 115 that can be adapted to output the generated breakdown products and/or to provide control signals for controlling a production process of the production system 120 based on the generated breakdown products.

The digital representation providing unit 111 is adapted to provide a digital representation indicative of chemical material physicochemical parameters, of a chemical material, in particular a polymer or a functional chemical compound for which breakdown products should be generated. The digital representation providing unit 111 can refer, for instance, to an input unit into which a user can input the respective digital representation. Moreover, the digital representation providing unit 111 can refer to or be part of a user interface that allows the user to interact with the apparatus 110 and/or the database 140. However, the digital representation providing unit 111 can also refer to or be communicatively coupled with a storage unit on which the digital representation of the polymer is already stored. Generally, the digital representation can directly comprising the polymer physicochemical parameters that are indicative of parameters quantifying physicochemical characteristics of the respective polymer. However, instead of directly providing the polymer physicochemical parameters also a synthesis specification of the polymer can be provided. In this case, it is preferred that the digital representation providing unit 111 is further adapted to determine the polymer physico-chemical parameters from the synthesis specification. In particular, it is preferred that the digital representation providing unit 111 is adapted to identify from the synthesis specification types and amounts of subgroups of the polymer and to determine the polymer physicochemical parameters based on the identified types and amounts of subgroups. In particular, the digital representation providing unit 111 can be adapted to determine for each identified subgroup respective subgroup physicochemical parameters, for instance, by accessing a database on which for a plurality of the most relevant subgroups respective physicochemical parameters are stored. The physicochemical parameters of the polymer can then be determined based on the subgroup physicochemical parameters of the subgroups and Optionally, also on the determined amount and type of the sub-groups, for example, by weighted averaging of the subgroup physicochemical parameters of the subgroups. The digital representation providing unit 111 is then adapted to provide the digital representation comprising the polymer physicochemical parameters, for instance, to the determination unit 114.

The habitat providing unit 112 is adapted to provide the biodegradation habitat indicative of an enzyme environment in the biodegradation habitat. The habitat providing unit 112 can refer, for instance, to an input unit into which a user can input a respective biodegradation habitat. For example, a user interface can be provided that allows a user to select from a number of predetermined biodegradation habitats. In a preferred embodiment, the habitat providing unit can be communicatively coupled to or refer to a user interface that allows to indicate a geolocation, for instance, by marking a location on a map, by indicating coordinates, or providing a name of a region, for in-stance, a political or geological region, wherein the habitat providing unit can then be adapted to provide a biodegradation habitat based on the geolocation. For example, if the geolocation indicates a specific sea region like the North Sea or the Atlantic, the habitat providing unit can be adapted to determine as biodegradation habitat a marine habitat.

Generally, a biodegradation habitat is indicative of habitat property values of habitat descriptors influencing a biodegradation of a chemical material in the respective habitat. In particular, habitat properties are indicative of environmental characteristics of the habitat, for example, for a marine habitat a salt concentration can strongly influence the biodegradation of a polymer in the marine habitat. Generally, since the breakdown products are generated, the chemical influence of the habitat properties on the polymer may be important for this application. Thus, it is the influence of the habitat properties on the biology of the habitat, in particular, on the microbial population and/or the enzymes of the habitat, that may be provided by the habitat providing unit. The habitat providing unit may refer for instance, to an input unit into which a user can input a respective biodegradation habitat by inputting enzymes and/or microbes producing enzymes in the biodegradation habitat.

The model providing unit 113 is adapted to provide a breakdown model based on the provided biodegradation habitat. In particular, it is preferred that the model providing unit 113 is adapted to select the breakdown model from a plurality of breakdown models stored already on a database. For example, a breakdown model can be trained with respect to training data corresponding to one or more specific biodegradation habitats, indicative of an enzyme environment and/or microbes in the biodegradation habtiat. These specific biodegradation habitats can be defined with respect to habitat property values or value ranges that define for which biodegradation habitat the respective breakdown model is suitable. For example, a lookup table can be provided that allows the model providing unit to select based on the biodegradation habitat, for in-stance, based on the habitat property values of the biodegradation habitat, which of the breakdown models is suitable. However, the model providing unit 113 can also comprise or refer to an input unit to which the breakdown model can be received, for instance, by a user selection or user input that indicates which biodegradation model should be used.

The breakdown model may an enzymatic pathway model or a data-driven model. The data driven model may relate a chemical material and the biodegradation habitat to breakdown products. In particular the data driven model may be parametrized based on a chemical material, biodegradation breakdown products and enzymes. In an alternative, the data driven model may be parametrized based on a chemical product, biodegradation breakdown products, and microbes. In a further alternative, the data driven model may be parametrized based on breakable bonds in the chemical material, biodegradation breakdown products and microbes. In a further alternative, the data driven model may be parametrized based on breakable bonds in the chemical material, biodegradation breakdown products and enzymes.

In a preferred embodiment, the data-driven model may refer to a machine learning model, for instance, utilizing regression model based algorithms or classifier model based algorithms. A regression model based algorithm can be based on any of a neural network algorithm, a Linear Regression algorithm, a LASSO algorithm, a Ridge Regression algorithm, a MARS algorithm, a Random Forest algorithm, and a Boosted Trees algorithm. A classifier based model algorithm can be based on any of a Random Forest algorithm, a Logistic Regression algorithm, and a SVM algorithm. The inventors have found that for most applications, in particular, Linear Regression, Random Forest, Neural Network and MARS based algorithms are suitable.

The breakdown model can be trained, for instance, utilizing training apparatus 130. In particular, the training apparatus 130 comprises a training data providing unit 131 for providing training data for training the data-driven based biodegradation model.

Further, the training apparatus 130 may comprise a model providing unit 132 adapted to provide a data-driven based trainable breakdown model, for instance, a breakdown model comprising parameters that can be set during the training process for training the breakdown model. For example, a trainable breakdown model may already be stored on a storage unit to which the model providing unit 132 can have access for providing the same. Moreover, the training apparatus 130 may comprise a training unit 133 for training the provided data-driven based breakdown model based on the provided training data. In particular, the training can refer to varying the parameters of the breakdown model based on the respective training data until the breakdown model is adapted to generate breakdown products chemical material based on a digital representation. Generally, any known training algorithms for training data-driven, in particular, machine learning based models can be utilized.

The training apparatus 130 then may comprise a trained model providing unit 134 that is adapted to provide the trained breakdown model, for instance, to a storage unit on which respectively trained breakdown models for different habitat and/or different types of chemical materials are stored. However, the trained model providing unit 134 can also be adapted to directly provide the trained breakdown model, for instance, to the breakdown model providing unit 113 of apparatus 110.

In all cases, the breakdown model providing unit 113 is then adapted to provide a suitable trained breakdown model to the property determination unit 114. The generating unit 114 can then utilize the breakdown model and the provided digital representation for generating the breakdown products. In particular, the generating unit 114 can be adapted to utilize the polymer physicochemical parameters indicated by the digital representation as input to the breakdown model that has, as already described above, been trained to then provide as output a determination for the breakdown products for which it has been trained. An output unit referring, for instance, to a display, can then be adapted to output the generated breakdown products. However, the output unit can additionally or alternatively be adapted to provide the breakdown products to a data-base 140 for storing the chemical material in association with the generated breakdown products for future usage.

Optionally the apparatus 110 may comprise a biodegradation determining unit 117 adapted to determine biodegradability of the chemical material based on the breakdown products. In an alternative, a data driven biodegradation model may be provided by database 140. The data driven biodegradation model may be parametrized based on breakdown products and their respective biodegradability- or the respective biodegradability of the chemical material. Biodegradability of the chemical material may be determined based on the breakdown products and the data driven biodegradation model. In an alternative, the breakdown model may be a enzymatic pathway model. The biodegradation determining unit may then be adapted to determine biodegradability based on the breakdown products or of the breakdown products, if inert breakdown products remain in the pathway.

The biodegradation determining unit may be coupled to the output and/or control unit 115. Biodegradability of the chemical material may then be provided via output unit 115.

Optionally, the apparatus 110 may comprise an ecotoxicology determining unit 118 adapted to determine toxicity of the chemical material based on the breakdown products or of the breakdown products. In an alternative, a data driven ecotox model may be provided by database 140. The data driven ecotox model may be parametrized based on breakdown products and their respective toxicity. Toxicity of the chemical material may be determined based on the breakdown products and the data driven ecotox model. In an alternative, the ecotox model may be an enzymatic pathway model, describing metabolism of the breakdown products.

The ecotoxicology determining unit 118 may be coupled to the output and/or control unit 115. Biodegradability or ecotoxicology of the chemical material may then be provided via output unit 115.

Optionally, the apparatus 110 can comprise the control unit 115 that is adapted to provide control and/or monitoring signals based on the determined biodegradability and/or toxicology for controlling a production process of a production system 120. In particular, it is preferred that the control unit 115 is adapted to receive a target biodegradability and/or target toxicology for a polymer and to compare the received target biodegradability and/or toxicology with the determined biodegradability and/or toxicology and to provide the control and/or monitoring signal depending on the comparison, Optionally, to provide control and/or monitoring signals that indicate the usage or production of the chemical material, such as the polymer, for which the biodegradability and/or toxicology has been determined. Moreover, the control and/or monitoring signals can be indicative of a machine executable synthesis specification of the chemical material, such as polymer, for which the biodegradability and/or toxicology has been determined, when the result of the comparison refers to the determined biodegradability being within a predetermined range around the target biodegradability and/or the result of the comparison refers to the determined toxocology being within a predetermined range around the target toxicology. However, the control unit 115 can also be adapted to control and/or monitor the production process of another chemical material based on the determined biodegradability and/or toxicology, for instance, to provide control signals indicative of a machine executable synthesis specification for another chemical material utilizing or comprising the respective chemical material. Moreover, the control unit 115 can provide control and/or monitoring signals for controlling and/or moniotirng a habitat for biodegrading a polymer, for instance, in a waste management facility. For example, a target biodegradability can be met for specific habitat descriptors and the control unit 115 can then be adapted to provide control signals that control the facility such that these habitat properties values are met.

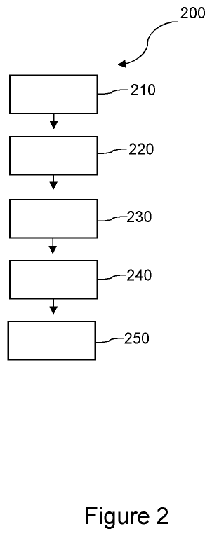

FIG. 2 shows schematically and exemplarily a flow chart of a method for generating breakdown products of a chemical material, in particular of a polymer or a functional chemical compound.

The method 200 comprises a first step 210 of providing a digital representation of the chemical material. In particular, the providing of the digital representation in this step can be in accordance with the principles described above with respect to the digital representation providing unit 111. Further, in a step 220, a biodegradation habitat indicative of habitat property values of habitat descriptors influencing a biodegradation of chemical material in a respective habitat may be provided. In particular biodegradation habitat properties indicative of the microbial environment and/or enzymes may be provided. Also for this step 220, the principles described above, for instance, with respect to the habitat providing unit 112, can be applied. Further, in step 230, a breakdown model is provided that is adapted to determine the breakdown products of the chemical material based on the digital representation. As already discussed above in more detail, the providing of the break down model can also refer to a selection of the break down model based on the provided biodegradation habitat. Moreover, the break down model is a data driven model parameterized with respect to the biodegradation habitat such that it can determine breakdown products of a chemical material based on the digital representations. Generally, the steps 210, 220 and 230 can be performed in arbitrary order or even concurrently. In a following step 240, the breakdown products may be generated based on the provided digital representation of the chemical material and the breakdown model. In an optional step 250, the breakdown products can then be provided, for instance, to a user interface such that the determined breakdown products for the chemical material can be displayed on a display. In an optional step 251 biodegradation of the chemical material may be determined as described with reference to FIG. 1. In an optional step 252, toxicology of the chemical material and/or the breakdown products may be determined as described in more detail with respect to FIG. 1.

However, in step 250, the method can additionally or alternatively comprise generating control and/or monitoring signals that allow for a controlling and/or monitoring of a production process of a product, for instance, the chemical material and/or a product comprising the chemical material, as already described above in more detail.

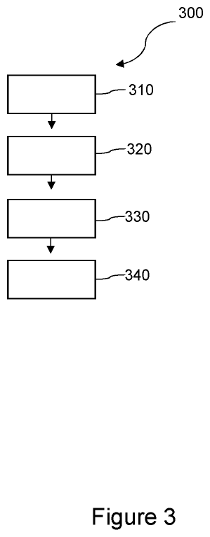

FIG. 3 shows schematically and exemplarily a flow chart of a method for training the data driven based breakdown model utilized, for instance, in the method 200 discussed with respect to FIG. 2.

Generally, the method 300 can be performed, for instance, by respective units of the training apparatus 130 as described with respect to FIG. 1. The method 300 comprises a step 310 of providing training data for training the data driven based break down model. The training data comprises a) digital representation of a plurality chemical materials, and b) breakdown products associated with each training chemical material in a respective biodegradation habitat indicative of an enzyme environment in the biodegradation habitat, the training data can be provided in accordance with the principles described above with respect to the training data providing unit 131 described with respect to FIG. 1. The method comprises further a step 320 of providing a data driven based trainable breakdown model, for instance, a machine learning based break down model like a neural network. Generally, the step 310 and the step 320 can be performed in arbitrary order or even at the same time. The method 300 then further comprises a step 330 of training the provided data driven based breakdown model based on the provided training data, for instance, by varying parameters in the data driven based trainable breakdown model, such that the trained breakdown model is adapted to generate breakdown products of a chemical material based on a digital representation of the chemical material. In step 340 the trained breakdown model can then be provided, for instance, by storing the trained biodegradation model on a storage or by directly providing the trained breakdown model to the apparatus 130 as described with respect to FIG. 1.

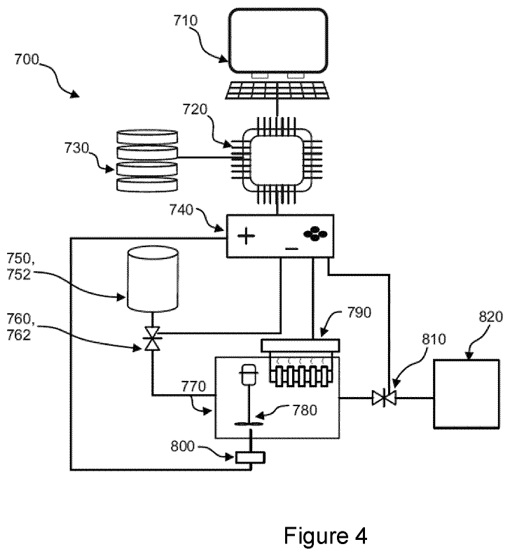

FIG. 4 shows an exemplary system 700 for producing a chemical product based on a synthesis specification generated according to the invention.

In this example the system comprises a user interface 710 and a processor 720, associated with a control unit 740. The user interface 710 and the processor 720 can be associated with or realized in accordance with the principles described above, in particular, can be adapted to perform a computer implemented method to determine a target polymer and/or synthesis specification based on a determined biodegradability, as described above. The control unit 740 is, for example, configured for receiving control data generated according to the invention as described above, in particular, to receiving control data generated based on a synthesis specification of a polymer comprising a target biodegradability. In this example the control data is provided from a data base 730, in other examples, however the control data can also be provided from a server or any other computational unit for distributing data. Vessels 750, 752 each contain a component of the chemical product, for example, pre-polymers, catalysts, etc. In general more than two vessels are present, however, in this example for illustrative purposes only two are shown. Valves 760, 762 are associated with vessels 750, 752. Valves 750 and 752 can be controlled to dose appropriate amounts of each component into reactor 770, according to the synthesis specification. A motor 800 of a mixer 780 may also be controlled by the control unit according to the synthesis specification. An optional heater 790 may also be controlled according to the synthesis specification. Finally, an exit valve 810 in fluid communication with the reactor may be controlled by the control unit to provide the chemical product to a container or test system 820.

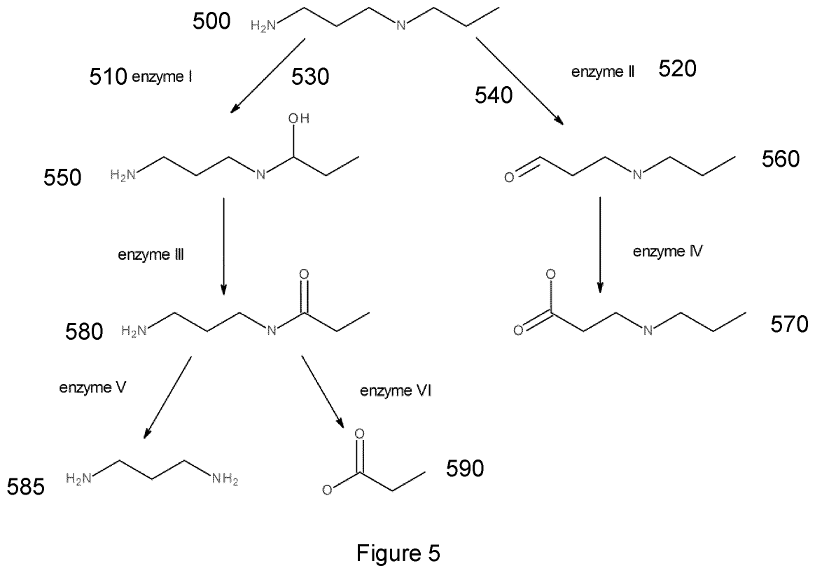

FIG. 5 shows schematically and exemplarily a biodegradation path of a molecule 500, here NPropyl-1,3-Propanediamine. The enzymatic pathway is dominated by enzyme I 510 and enzyme II 520. Enzyme I, induces a catabolic reaction 530 leading to a first initial breakdown product 540. Enzyme 2 induces a second catabolic reaction 550 leading to a second initial breakdown product 560. The second initial breakdown product may then be decomposed by enzyme IV, wherein enzyme IV induces a catabolic reaction resulting in a residual breakdown product 570 which in that example may not be further biodegradable. Consequently, molecule 500 is not fully biodegradable. The first initial breakdown product 540 may be further decomposed via enzyme Ill resulting in a secondary breakdown product 580. Enzymes V and VI may then decompose the secondary breakdown product further to generate breakdown products 585 and 590.

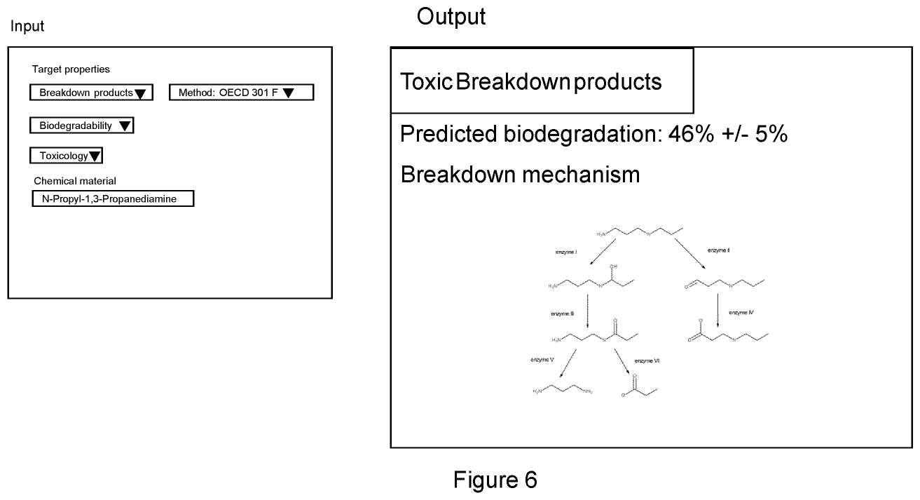

FIG. 6 shows exemplarily and schematically a possible user interface for interfacing, for example, with a processor performing the above-described method for generating breakdown products of a chemical material, e.g. of a polymer or a functional chemical compound.

In this example, an input screen is shown on the left. The input screen allows for a definition of a chemical material for which breakdown products should be generated. In this case as chemical material is n-propyl-1 3-propanediamine. The input screen allows in this example to input which application properties should be determined, in this case a biodegradability and a toxicology. An additional application property can for example be determined based on a respective prediction model for the respective additional application property or by any other known method. Further, the input screen can allow to input the type of biodegradation habitat indicative of an enzyme environment in the biodegradation habitat. However, this can also be omitted, if the habitat can be derived from other information, for example, as in this case from the selected measurement method for the biodegradability only applicable to waste water. Generally, the input can also refer to further information, for example, to defining habitat property values, intended applications, measurement method, etc. The user interface further comprises an output for displaying the breakdown products. In this example, the full enzymatic pathway of the breakdown process is displayed. For this example, it can be shown that a good prediction accuracy can be achieved when utilizing as polymer descriptor types a descriptor referring to a molar weight of the polymer, a descriptor referring to an amount of subgroups, and a descriptor referring to a hydrophiliy of the polymer. The values for such polymer descriptors can then be determined in accordance with the above-described principles for the defined polymer. An exemplary output screen is shown on the right of FIG. 6. In this case the output screen provides the result of the generation of breakdown products for chemical product and habitat defined on the input screen using one of the above-mentioned breakdown models. For this example, the result further states that the chemical material is not biodegradable and biodegradation or break down products are toxic.

The present disclosure has been described in conjunction with preferred embodiments and examples as well. However, other variations can be understood and effected by those persons skilled in the art and practicing the claimed invention, from the studies of the drawings, this disclosure and the claims.

Any steps presented herein can be performed in any order. The methods disclosed herein are not limited to a specific order of these steps. It is also not required that the different steps are performed at a certain place or in a certain computing node of a distributed system, i.e. each of the steps may be performed at different computing nodes using different equipment/data processing.

As used herein “determining” also includes “initiating or causing to determine”, “generating” also includes “initiating and/or causing to generate” and “providing” also includes “initiating or causing to determine, generate, select, send and/or receive”. “Initiating or causing to perform an action” includes any processing signal that triggers a computing node or device to perform the respective action.

In the claims as well as in the description the word “comprising” does not exclude other elements or steps and the indefinite article “a” or “an” does not exclude a plurality. A single element or other unit may fulfill the functions of several entities or items recited in the claims. The mere fact that certain measures are recited in the mutual different dependent claims does not indicate that a combination of these measures cannot be used in an advantageous implementation.

A single unit or device may fulfill the functions of several items recited in the claims. The mere fact that certain measures are recited in mutually different dependent claims does not indicate that a combination of these measures cannot be used to advantage.

Procedures like the providing of the functional chemical compound physicochemical parameters and the biodegradation model, the determining of the biodegradability, the providing of the biodegradability, etc. performed by one or several units or devices can be performed by any other number of units or devices. These procedures can be implemented as program code means of a computer program and/or as dedicated hardware.

A computer program product may be stored/distributed on a suitable medium, such as an optical storage medium or a solid-state medium, supplied together with or as part of other hardware, but may also be distributed in other forms, such as via the Internet or other wired or wireless telecommunication systems.

Any units described herein may be processing units that are part of a classical computing system. Processing units may include a general-purpose processor and may also include a field programmable gate array (FPGA), an application specific integrated circuit (ASIC), or any other specialized circuit. Any memory may be a physical system memory, which may be volatile, nonvolatile, or some combination of the two. The term “memory” may include any computer-readable storage media such as a non-volatile mass storage. If the computing system is distributed, the processing and/or memory capability may be distributed as well. The computing system may include multiple structures as “executable components”. The term “executable component” is a structure well understood in the field of computing as being a structure that can be software, hardware, or a combination thereof. For instance, when implemented in software, one of ordinary skill in the art would understand that the structure of an executable component may include software objects, routines, methods, and so forth, that may be executed on the computing system. This may include both an executable component in the heap of a computing system, or on computer-readable storage media. The structure of the executable component may exist on a computer-readable medium such that, when interpreted by one or more processors of a computing system, e.g., by a processor thread, the computing system is caused to perform a function. Such structure may be computer readable directly by the processors, for instance, as is the case if the executable component were binary, or it may be structured to be interpretable and/or compiled, for instance, whether in a single stage or in multiple stages, so as to generate such binary that is directly interpretable by the processors. In other instances, structures may be hard coded or hard wired logic gates, that are implemented exclusively or near-exclusively in hardware, such as within a field program-mable gate array (FPGA), an application specific intograted circuit (ASIC), or any other specialized circuit. Accordingly, the term “executable component” is a term for a structure that is well understood by those of ordinary skill in the art of computing, whether implemented in software, hardware, or a combination. Any embodiments herein are described with reference to acts that are performed by one or more processing units of the computing system. If such acts are implemented in software, one or more processors direct the operation of the computing system in response to having executed computer-executable instructions that constitute an executable component. Computing system may also contain communication channels that allow the computing system to communicate with other computing systems over, for example, network. A “network” is defined as one or more data links that enable the transport of electronic data between computing systems and/or modules and/or other electronic devices. When information is transferred or provided over a network or another communications connection, for example, either hardwired, wireless, or a combination of hardwired or wireless, to a computing system, the computing system properly views the connection as a transmission medium. Trans-mission media can include a network and/or data links which can be used to carry de-sired program code means in the form of computer-executable instructions or data structures and which can be accessed by a general-purpose or special-purpose computing system or combinations. While not all computing systems require a user interface, in some embodiments, the computing system includes a user interface system for use in interfacing with a user. User interfaces act as input or output mechanism to users for instance via displays. Those skilled in the art will appreciate that at least parts of the invention may be practiced in network computing environments with many types of computing system configurations, includeing, personal computers, desktop computers, laptop computers, message processors, handheld devices, multi-processor systems, microprocessor-based or programmable consumer electronics, network PCs, minicomputers, main-frame computers, mobile telephones, PDAs, pagers, routers, switches, datacenters, wearables, such as glasses, and the like. The invention may also be practiced in distributed system environments where local and remote computing system, which are linked, for example, either by hardwired data links, wireless data links, or by a combination of hardwired and wireless data links, through a network, both perform tasks. In a distributed system environment, program modules may be located in both local and remote memory storage devices.

Those skilled in the art will also appreciate that at least parts of the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources, e.g., networks, servers, storage, applications, and services. The definition of “cloud computing” is not limited to any of the other numerous advantages that can be obtained from such a model when deployed. The computing systems of the figures include various components or functional blocks that may implement the various embodiments disclosed herein as explained. The various components or functional blocks may be implemented on a local computing system or may be implemented on a distributed computing system that includes elements resident in the cloud or that implement aspects of cloud computing. The various components or functional blocks may be implemented as software, hardware, or a combination of soft-ware and hardware. The computing systems shown in the figures may include more or less than the components illustrated in the figures and some of the components may be combined as circumstances warrant.

Any disclosure and embodiments described herein relate to the methods, the systems, devices, the computer program element lined out above and vice versa. Advantageously, the benefits provided by any of the embodiments and examples equally apply to all other embodiments and examples and vice versa.

All terms and definitions used herein are understood broadly and have their general meaning.

1. A computer implemented method for generating breakdown products of a chemical material comprising:
providing a target chemical material;
providing a biodegradation habitat, indicative of an enzyme environment in the biodegradation habitat
providing a breakdown model, relating a chemical material and the biodegradation habitat to breakdown products
generating breakdown products based on the provided target chemical material and the breakdown model; and
providing the breakdown products.

2. The method according to claim 1, wherein the method further comprises providing a biodegradation test method, wherein the provided biodegradation test method is indicative of a standardised biodegradation test method for determining experimentally a biodegradation of a chemical material, wherein the enzyme environment is selected based on the provided biodegradation test method.

3. The method according to claim 1, wherein the biodegradation habitat refers to any one of a marine habitat, a waste water habitat, a limnic habitat, a compost habitat, an anaerobic habitat or a soil habitat.

4. The method according to claim 3, wherein the biodegradation habitat refers to a marine habitat and wherein the habitat descriptors refer to at least one of a salt concentration, a sedimentation type, oxygen level, location, sample depth, a water temperature, a nutrient concentration, a pH value, an environmental type and the enzyme environment.

5. The method according to claim 3, wherein the biodegradation habitat refers to waste water and the habitat descriptors refer to at least one of a water temperature, a microbial community, a sludge concentration, a nutrient concentration, a pH value, a test duration and the enzyme environment.

6. The method according to claim 3, wherein the biodegradation habitat refers to soil and the habitat descriptors refer to at least one of a temperature, a sand content, a pH value, a moisture content, a nutrient concentration, a microbial community and the enzyme environment.

7. The method according to claim 3, wherein the biodegradation habitat refers to compost and the habitat descriptors refer to at least one of a temperature, compost activity, a pH value, a moisture content, humidity, compost maturity, compost composition, compost origin, a nutrient concentration, a microbial community and an enzyme environment.

8. The method according to claim 1, wherein habitat property values for the habitat descriptors are stored associated with respective geolocations, wherein the providing of a biodegradation habitat refers to providing a geolocation of the habitat and retrieving the habitat property values for the geolocation from storage.

9. The method according to claim 1, wherein the breakdown model may be a data driven model.

10. The method according to claim 9, wherein the break down model is trained based on historic measurement data relating to the biodegradation process and/or the habitat of the biodegradation process.

11. The method according to claim 9, wherein the training data may include the representation of the chemical material, the representation of the habitat the chemical material was exposed to and one or more fragment(s) of the chemical material measured at one or more time interval(s).

12. The method according to claim 1, wherein the breakdown model may be an enzymatic pathway model.

13. The method according to claim 1, wherein toxicology of the chemical material is determined based on an ecotox model relating breakdown products to their toxicology.

14. An apparatus for generating breakdown products of a target chemical material, wherein the apparatus comprises:
a digital representation providing unit for providing the chemical material,
a habitat providing unit for providing a biodegradation habitat, wherein the biodegradation habitat is indicative of an enzyme environment in the biodegradation habitat,
a model providing unit for providing a breakdown model habitat, wherein the biodegradation model is adapted to generate breakdown products of a chemical material in the respective biodegradation habitat, wherein the biodegradation model is a data driven model or an enzymatic pathway model, and
a generating unit for generating the breakdown products of the chemical material based on the breakdown model, the provided biodegradation habitat and the chemical material.

15. A computer program element with instructions, which when executed on a processing device is configured to carry out the steps of the method of claim 1.
